(19) United States
(12) Patent Application Publication
(10) Pub. No.: US 2025/0000168 A1
(43) Pub. Date: Jan. 2, 2025

(54) VERIFYING OPERATION OF TEMPERATURE SENSOR OF AN AEROSOL-GENERATING DEVICE

(71) Applicant: Philip Morris Products S.A., Neuchatel (CH)

(72) Inventors: Michel BESSANT, Neuchâtel (CH); Johannes Petrus Maria PIJNENBURG, Neuchâtel (CH); Fabrice STEFFEN, Colombier (CH)

(73) Assignee: Philip Morris Products S.A., Neuchatel (CH)

(21) Appl. No.: 18/709,830

(22) PCT Filed: Nov. 23, 2021
(86) PCT No.: PCT/CN2021/132394

§ 371 Date: May 14, 2024

(51) Int. Cl. A24F 40/57; A24F 40/46; A24F 40/51

(52) U.S. Cl. CPC A24F 40/57; A24F 40/46; A24F 40/51

(57) ABSTRACT

An aerosol-generating device for generating an aerosol from an aerosol-forming substrate is provided, the aerosol-generating device including: an electrically powered heater element configured to heat the aerosol-forming substrate; a temperature sensor coupled to the electrically powered heater element and configured to sense a temperature of the electrically powered heater element; a power source configured to supply power to the electrically powered heater element and the temperature sensor; and control electronics configured to measure an impedance value of the electrically powered heater element, correlate the measured impedance value with a value of impedance determined from the temperature of the electrically powered heater element as sensed by the temperature sensor, and control the supply of power to the electrically powered heater element based on the correlation. A method of controlling an electrically powered heater element of the aerosol-generating device is also provided.

The present disclosure relates to verifying correct operation of a temperature sensor of an aerosol-generating device. Conversely, the present disclosure also relates to detecting incorrect operation of a temperature sensor of an aerosol-generating device.

Aerosol-generating devices which generate an aerosol by heating rather than burning an aerosol-forming substrate are known. Such aerosol-generating devices employ an electrically-powered heater element which is controlled in accordance with a heating profile defining a target operating temperature for the heater element over a usage session. To provide a satisfactory user experience, it is important to control the heater element such that its temperature tracks the target operating temperature defined by the heating profile as accurately as possible. Such accurate tracking requires correspondingly accurate determination of the heater element temperature. Known aerosol-generating devices employ a temperature sensor which may be coupled to the heater element by adhesive or similar means of coupling. However, failure of the coupling between the temperature sensor and heater element may result in detachment of the temperature sensor from the heater element. The detachment of the temperature sensor would result in the temperature sensor sensing temperatures which are lower than the actual temperature of the heater element. In these circumstances, the temperature communicated from the temperature sensor is likely to be lower than the target operating temperature defined for the heater element by the heating profile. The control methodology employed by such known aerosol-generating devices is based on an assumption that the temperature sensor is accurately sensing the temperature of the heater element. So, the control methodology would respond to the lower than expected temperature reading from the detached temperature sensor by increasing the supply of power to the heater element. The increase in the supply of power based on the erroneous reading from the detached temperature sensor would be likely to result in overheating of the heater element, as well as more rapid depletion of the power source of the aerosol-generating device. Other faults connected with the structure or operation of the temperature sensor may lead to a divergence between the temperature sensed by the temperature sensor and the actual temperature to be detected, such as the temperature of the heater element.

It is therefore desirable to provide a methodology for verifying satisfactory operation and/or detecting unsatisfactory operation of a temperature sensor of an aerosol-generating device.

According to a first aspect of the present disclosure, there is provided an aerosol-generating device for generating an aerosol from an aerosol-forming substrate. The aerosol-generating device comprises an electrically-powered heater element for heating the aerosol-forming substrate; a temperature sensor coupled to the heater element and configured to sense the temperature of the heater element; a power source configured to supply power to the heater element and the temperature sensor; and control electronics. The control electronics are configured to measure an impedance value of the heater element; correlate the measured impedance value with a value of impedance determined from the temperature of the heater element as sensed by the temperature sensor; and control the supply of power to the heater element based on the correlation.

Divergence between i) the measured impedance value and ii) the value of impedance determined from the temperature of the heater element as sensed by the temperature sensor may indicate a fault with the operation of the temperature sensor and/or the positioning of the temperature sensor relative to the heater element.

The heater element may be an electrically resistive heating element. By way of example, the heater element may comprise one or a plurality of heating tracks. The heating tracks may be made from stainless steel having a thickness of about 50 micrometres, or preferably about 25 micrometres. Alternatively, the heating tracks may be made from inconel having a thickness of about 50.8 micrometres, or about 25.4 micrometres. In further alternatives, the heating tracks may be made from copper having a thickness of about 35 micrometres, or from constantan having a thickness of about 25 micrometres, or from nickel having a thickness of about 12 micrometres, or from brass having a thickness of about 25 micrometres.

The temperature sensor may be a resistance temperature detector, such as a Pt100 or Pt1000 temperature sensor. However, other forms of temperature sensor/resistance temperature detector may be employed.

The measured impedance value may be derived from measurements of a voltage and a current applied to the heater element.

This paragraph provides an exemplary illustration of how the measured impedance value may be correlated with the value of impedance determined from the temperature of the heater element as sensed by the temperature sensor. Where the heater element is an electrically resistive heater element and no inductive aspects are present, the impedance of the heater element may be regarded as being analogous to its resistance; in this scenario, the terms impedance and resistance may be used interchangeably. Where a current, Iₕₑₐₜₑᵣ and a voltage. Vₕₑₐₜₑᵣ are applied to the heater element, the impedance or resistance, Rₕₑₐₜₑᵣ of the heater element may be related to the current and voltage as follows:

$${R}_{heater} = \frac{{V}_{heater}}{{I}_{heater}} \qquad Equation 1$$

The impedance or resistance, Rₕₑₐₜₑᵣ, may be measured indirectly through knowledge or measurement of the voltage, Vₕₑₐₜₑᵣ and the current, Iₕₑₐₜₑᵣ.

The impedance or resistance, Rₕₑₐₜₑᵣ of an electrically resistive heater element may be related to the temperature of the heater element. Accordingly, the impedance or resistance, Rₕₑₐₜₑᵣ of the heater element may also be expressed as:

$${R}_{heater} ( T ) = {R}_{0} + {R}_{0} \times const \times T \qquad Equation 2$$

The terms in Equation 2 above have the following meaning: T represents the temperature of the heater element as sensed by the temperature sensor; R₀ represents the resistance of the heater element at a temperature T having a value of zero; “const” is a numerical constant whose value is dependent on the characteristics of the specific heater element being used in the aerosol-generating device (for example, the material from which the heater element is made) and may typically be supplied by the manufacturer of the heater element.

In a first scenario in which the temperature sensor is correctly positioned and correctly functioning, it would be expected that the impedance or resistance, Rₕₑₐₜₑᵣ provided by Equation 1 would be the same or very close in value to that provided by Equation 2. However, in the event of a fault with the positioning or operation of the temperature sensor, a mismatch would be develop between the values of impedance or resistance, Rₕₑₐₜₑᵣ provided by both equations. In this way, it can be seen how the correlation between i) the measured impedance value of the heater element and ii) the value of impedance determined from the temperature of the heater element as sensed by the temperature sensor, may be used to provide an indication of whether the temperature sensor is correctly positioned and correctly functioning.

Preferably, the control electronics may be configured to compare the measured impedance value with the determined impedance value. The control electronics may also be configured to reduce or terminate the supply of power to the heater element if the magnitude of a difference between the measured impedance value and the determined impedance value exceeds a predetermined threshold. In this manner, the control electronics may take corrective action where the correlating of the measured impedance value with the determined impedance value indicates an unacceptable degree of divergence. The corrective action may help to reduce the likelihood of overheating of the heater element. Further, the corrective action may also help to reduce the risk of the power source being prematurely depleted of energy.

Conveniently, the predetermined threshold may be between 0.05 ohm and 1 ohm, or between 0.05 ohm and 0.5 ohm, or between 0.05 ohm and 0.2 ohm, or between 0.1 and 0.15 ohm. Other values may be selected for the predetermined threshold depending on the degree of sensitivity which is desired for detecting potential faults in the positioning or operation of the temperature sensor.

A difference between the measured impedance value and the determined impedance value may be indicative of a difference between the actual temperature of the heater element and the temperature as sensed by the temperature sensor. Accordingly, the magnitude of the predetermined threshold may be set to correspond with what is deemed an acceptable maximum divergence between the temperature as sensed by the temperature sensor and the actual temperature.

The control electronics may be configured to compare the measured impedance value with the determined impedance value. The control electronics may also be configured to reduce or terminate the supply of power to the heater element if the measured impedance value differs in magnitude from the determined impedance value by more than 5% of the determined impedance value, or by more than 2.5% of the determined impedance value, or by more than 1% of the determined impedance value, or by more than 0.5% of the determined impedance value. Alternatively, the control electronics may be configured to reduce or terminate the supply of power to the heater element if the determined impedance value differs in magnitude from the measured impedance value by more than 5% of the measured impedance value, or by more than 2.5% of the measured impedance value, or by more than 1% of the measured impedance value, or by more than 0.5% of the measured impedance value.

Advantageously, the temperature sensor may have a resistivity dependent on the temperature of the heater element, and the control electronics configured to measure a voltage associated with the temperature sensor, the voltage being dependent on the resistivity of the heater element. The control electronics may comprise or be communicably coupled to a memory storing pre-configured data, the pre-configured data comprising a plurality of voltage values and a corresponding plurality of temperature values. The control electronics may further be configured to correlate the measured voltage with the pre-configured data and determine the temperature of the heater element based on the correlation between the measured voltage and the pre-configured data. The determined heater element temperature resulting from this correlation between the measured voltage and the pre-configured data may be used as representing the temperature of the heater element as sensed by the temperature sensor (corresponding to the temperature, T, of Equation 2 above).

The correlation of the measured voltage with the pre-configured data of voltage and corresponding temperature values may allow the heater element temperature corresponding to the measured voltage to be determined with reduced computational burden and complexity.

The pre-configured data may be in the form of a look-up table. The look-up table may comprise the plurality of voltage values and corresponding plurality of temperature values. The control electronics may be configured to associate the measured voltage to the voltage value in the look-up table closest in magnitude to the measured voltage, and to the temperature value in the look-up table corresponding to that voltage value. The control electronics may then use the temperature value in the look-up table associated with the measured voltage as the determined temperature of the heater element. In this manner, the control electronics may efficiently determine the temperature of the heater element.

Advantageously, the control electronics may also be configured to compare the determined temperature of the heater element with a target temperature for the heater element. The control electronics may further be configured to adjust the supply of power from the power source to the heater element so as to reduce any difference between the determined temperature of the heater element and the target temperature for the heater element. In this manner, the heater element temperature may better track the target temperature for the heater element. A feedback loop or similar means may be employed to perform this comparison.

Preferably, the control electronics is configured to control the supply of power to the heater element according to a heating profile, in which the heating profile defines the target temperature for the heater element over a usage session. The heating profile may be stored in a memory forming part of or communicably coupled to the control electronics; this memory may be the same as or different to the memory which stores the pre-configured data of voltage and temperature values.

The temperature sensor may be electrically coupled to a resistor, the resistor having a resistivity substantially invariant with temperature over a predetermined temperature range. The temperature sensor and the resistor may collectively form at least part of a resistor divider. The predetermined temperature range may be between 0 degrees Celsius and 425 degrees Celsius, or between 0 degrees Celsius and 400 degrees Celsius, or between 0 degrees Celsius and 375 degrees Celsius. Preferably, the resistivity being substantially invariant with temperature over the predetermined temperature range corresponds to the resistivity varying by no more than 15%, or by no more than 10%, or by no more than 5% over the predetermined temperature range.

The temperature sensor and the heater element may be disposed on opposed surfaces of an electrically-insulative substrate layer. The electrically-insulative substrate layer may be made from polyimide. The electrically-insulative substrate layer may be configured to withstand between 220 degrees Celsius and 320 degrees Celsius, preferably between 240 degrees Celsius and 300 degrees Celsius, preferably around 280 degrees Celsius. The electrically-insulative substrate layer may be made from Pyralux. The electrically-insulative substrate layer may be flexible, with a flexible substrate layer having an advantage that the layer can be rolled or formed into a desired shape; by way of example, the desired shape may be a tubular shape. The electrically-insulative substrate layer may comprise two or more sub-layers. Preferably, the electrically-insulative substrate layer may comprise a first portion and a second portion, the electrically-insulative substrate layer rolled into a tubular shape such that the heater element is disposed between the first and second portions of the electrically-insulative substrate layer. Conveniently, the temperature sensor may be disposed on an outward-facing surface of the electrically-insulative substrate layer.

The heater element may be disposed between distinct first and second electrically-insulative substrate layers. The temperature sensor may be disposed between the second electrically-insulative substrate layer and a third electrically-insulative substrate layer. The first electrically-insulative substrate layer, the heater element, the second electrically-insulative substrate layer, the temperature sensor and the third electrically insulative substrate layer may be successively laid over each other. Conveniently, an adhesive may be provided between each of the successive layers. A through hole may be provided through the thickness of the third electrically-insulative substrate layer to provide access to the temperature sensor. The first, second and third electrically-insulative substrate layers may be made from the same or different electrically-insulative materials; for example, polyimide as described in the preceding paragraph.

In a second aspect of the present disclosure, there is provided a method of controlling an electrically-powered heater element of an aerosol-generating device, the aerosol-generating device comprising a temperature sensor coupled to the heater element. The method comprises: measuring an impedance value of the heater element; correlating the measured impedance value with a value of impedance determined from the temperature of the heater element as sensed by the temperature sensor; and controlling a supply of power to the heater element based on the correlation.

The measuring of the impedance value of the heater element may comprise measuring a voltage and a current applied to the heater element.

Preferably, method may further comprise comparing the measured impedance value with the determined impedance value. Further, the method may also comprise reducing or terminating the supply of power to the heater element if the magnitude of a difference between the measured impedance value and the determined impedance value exceeds a predetermined threshold Conveniently, the predetermined threshold may be between 0.05 ohm and 1 ohm, or between 0.05 ohm and 0.5 ohm, or between 0.05 ohm and 0.2 ohm, or between 0.1 and 0.15 ohm.

The method may further comprise comparing the measured impedance value with the determined impedance value. The method may also comprise reducing or terminating the supply of power to the heater element if the measured impedance value differs in magnitude from the determined impedance value by more than 5% of the determined impedance value, or by more than 2.5% of the determined impedance value, or by more than 1% of the determined impedance value, or by more than 0.5% of the determined impedance value. Alternatively, the method may also comprise reducing or terminating the supply of power to the heater element if the determined impedance value differs in magnitude from the measured impedance value by more than 5% of the measured impedance value, or by more than 2.5% of the measured impedance value, or by more than 1% of the measured impedance value, or by more than 0.5% of the measured impedance value.

Advantageously, the temperature sensor may have a resistivity dependent on the temperature of the heater element. The method may further comprise measuring a voltage associated with the temperature sensor, the voltage being dependent on the resistivity of the heater element; correlating the measured voltage with pre-configured data comprising a plurality of voltage values and a corresponding plurality of temperature values; and determining the heater element temperature based on the correlation.

Advantageously, the method may further comprise comparing the determined temperature of the heater element with a target temperature for the heater element; and adjusting a supply of power to the heater element so as to reduce any difference between the determined temperature of the heater element and the target temperature for the heater element.

Advantageously, the method may further comprise comparing the determined temperature of the heater element with a target temperature for the heater element; and adjusting a supply of power to the heater element so as to reduce any difference between the determined temperature of the heater element and the target temperature for the heater element.

As used herein, the term “aerosol-generating device” is used to describe a device that interacts with an aerosol-forming substrate of an aerosol-generating article to generate an aerosol. Preferably, the aerosol-generating device is a smoking device that interacts with an aerosol-forming substrate of an aerosol-generating article to generate an aerosol that is directly inhalable into a user's lungs thorough the user's mouth. The aerosol-generating device may be a holder for a smoking article. Preferably, the aerosol-generating article is a smoking article that generates an aerosol that is directly inhalable into a user's lungs through the user's mouth. More preferably, the aerosol-generating article is a smoking article that generates a nicotine-containing aerosol that is directly inhalable into a user's lungs through the user's mouth.

As used herein, the term “aerosol-forming substrate” denotes a substrate consisting of or comprising an aerosol-forming material that is capable of releasing volatile compounds upon heating to generate an aerosol.

Preferably, the aerosol-forming substrate is a solid aerosol-forming substrate. However, the aerosol-forming substrate may comprise both solid and liquid components. Alternatively, the aerosol-forming substrate may be a liquid aerosol-forming substrate.

Preferably, the aerosol-forming substrate comprises nicotine. More preferably, the aerosol-forming substrate comprises tobacco. Alternatively or in addition, the aerosol-forming substrate may comprise a non-tobacco containing aerosol-forming material.

If the aerosol-forming substrate is a solid aerosol-forming substrate, the solid aerosol-forming substrate may comprise, for example, one or more of: powder, granules, pellets, shreds, strands, strips or sheets containing one or more of: herb leaf, tobacco leaf, tobacco ribs, expanded tobacco and homogenised tobacco.

Optionally, the solid aerosol-forming substrate may contain tobacco or non-tobacco volatile flavour compounds, which are released upon heating of the solid aerosol-forming substrate. The solid aerosol-forming substrate may also contain one or more capsules that, for example, include additional tobacco volatile flavour compounds or non-tobacco volatile flavour compounds and such capsules may melt during heating of the solid aerosol-forming substrate.

Optionally, the solid aerosol-forming substrate may be provided on or embedded in a thermally stable carrier. The carrier may take the form of powder, granules, pellets, shreds, strands, strips or sheets. The solid aerosol-forming substrate may be deposited on the surface of the carrier in the form of, for example, a sheet, foam, gel or slurry. The solid aerosol-forming substrate may be deposited on the entire surface of the carrier, or alternatively, may be deposited in a pattern in order to provide a non-uniform flavour delivery during use.

In a preferred embodiment, the aerosol-forming substrate comprises homogenised tobacco material. As used herein, the term “homogenised tobacco material” refers to a material formed by agglomerating particulate tobacco.

Preferably, the aerosol-forming substrate comprises a gathered sheet of homogenised tobacco material. As used herein, the term “sheet” refers to a laminar element having a width and length substantially greater than the thickness thereof. As used herein, the term “gathered” is used to describe a sheet that is convoluted, folded, or otherwise compressed or constricted substantially transversely to the longitudinal axis of the aerosol-generating article. Preferably, the aerosol-forming substrate comprises an aerosol former. As used herein, the term “aerosol former” is used to describe any suitable known compound or mixture of compounds that, in use, facilitates formation of an aerosol and that is substantially resistant to thermal degradation at the operating temperature of the aerosol-generating article.

Suitable aerosol-formers are known in the art and include, but are not limited to: polyhydric alcohols, such as propylene glycol, triethylene glycol, 1,3-butanediol and glycerine; esters of polyhydric alcohols, such as glycerol mono-, di- or triacetate; and aliphatic esters of mono-, di- or polycarboxylic acids, such as dimethyl dodecanedioate and dimethyl tetradecanedioate. Preferred aerosol formers are polyhydric alcohols or mixtures thereof, such as propylene glycol, triethylene glycol, 1,3-butanediol and, most preferred, glycerine.

The aerosol-forming substrate may comprise a single aerosol former. Alternatively, the aerosol-forming substrate may comprise a combination of two or more aerosol formers.

As used herein, the term “usage session” refers to a period in which a series of puffs are applied by a user to extract aerosol from an aerosol-forming substrate. The usage session may be a finite usage session; that is a usage session having a start and an end. The duration of the usage session as measured by time may be influenced by use during the usage session. The duration of the usage session may have a maximum duration determined by a maximum time from the start of the usage session. The duration of the usage session may be less than the maximum time if one or more monitored parameters reaches a predetermined threshold before the maximum time from the start of the usage session. By way of example, the one or more monitored parameters may comprise one or more of: i) a cumulative puff count of a series of puffs drawn by a user since the start of the usage session, and ii) a cumulative volume of aerosol evolved from the aerosol-forming substrate since the start of the usage session.

The invention is defined in the claims. However, below there is provided a non-exhaustive list of non-limiting examples. Any one or more of the features of these examples may be combined with any one or more features of another example, embodiment, or aspect described herein.

Examples will now be further described with reference to the figures, in which:

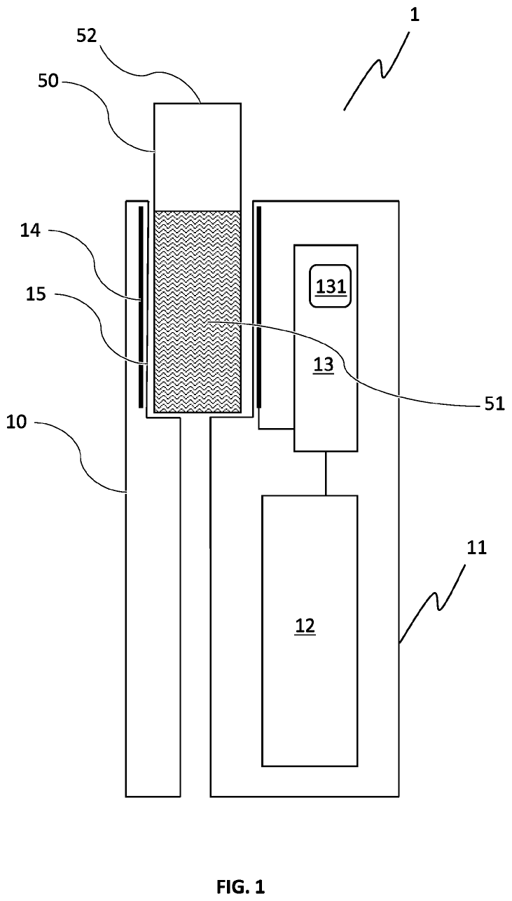

FIG. 1 shows an aerosol-generating system formed of an aerosol-generating device and an aerosol-generating article comprising an aerosol-forming substrate.

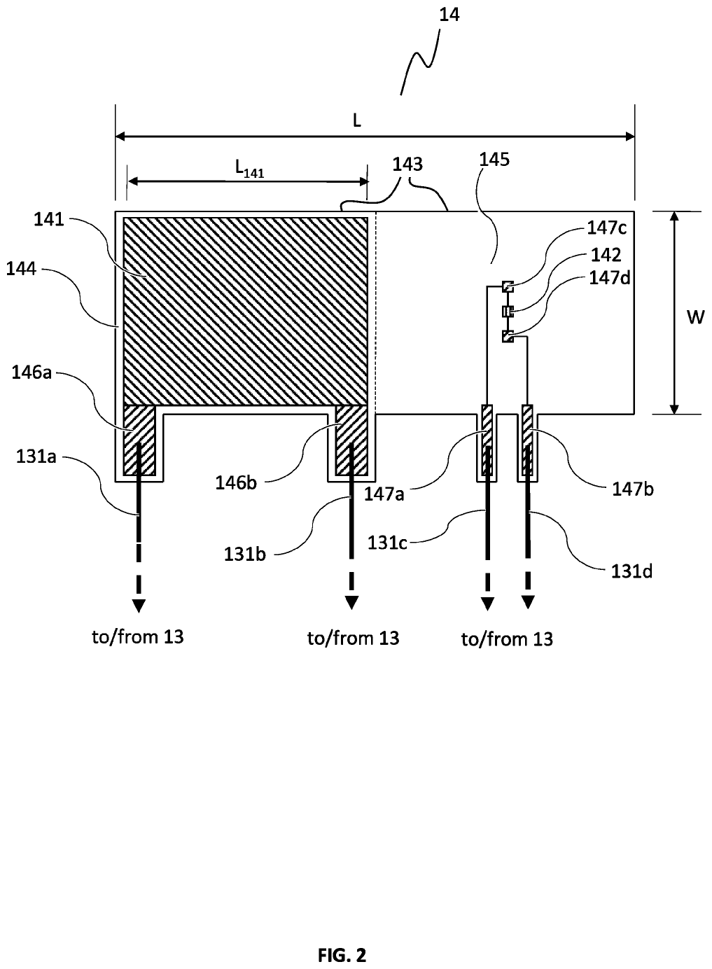

FIG. 2 shows the components of a first embodiment of a heating assembly of the aerosol-generating device of FIG. 1, before being rolled into a tubular shape.

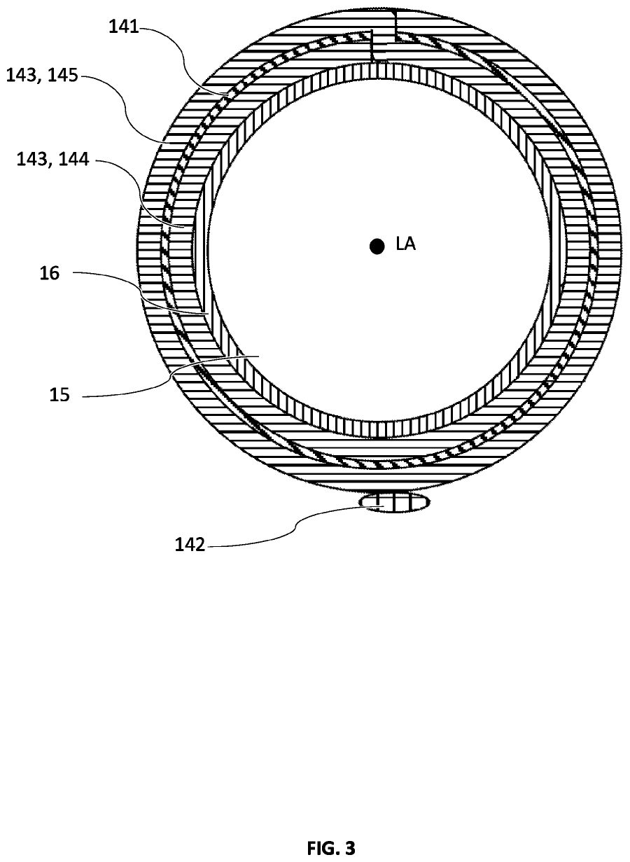

FIG. 3 shows an axial section view of the heating assembly of FIG. 2 after being rolled into a tubular shape.

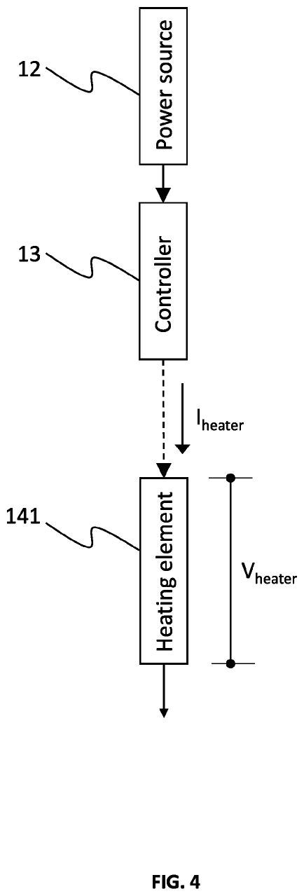

FIG. 4 shows how a controller of the aerosol-generating device of FIG. 1 controls the supply of power from a power source of the device so as to apply a current, Iₕₑₐₜₑᵣ and a voltage, Vₕₑₐₜₑᵣ to a heating element of the device.

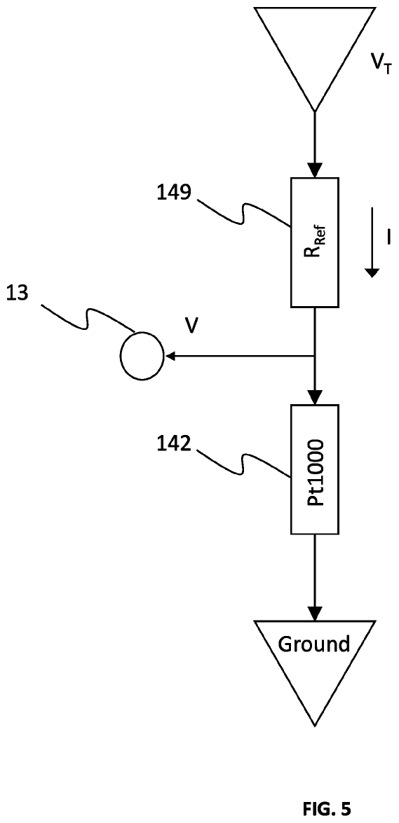

FIG. 5 illustrates how a voltage, V associated with a temperature sensor of the heating assembly of the aerosol-generating device is measured.

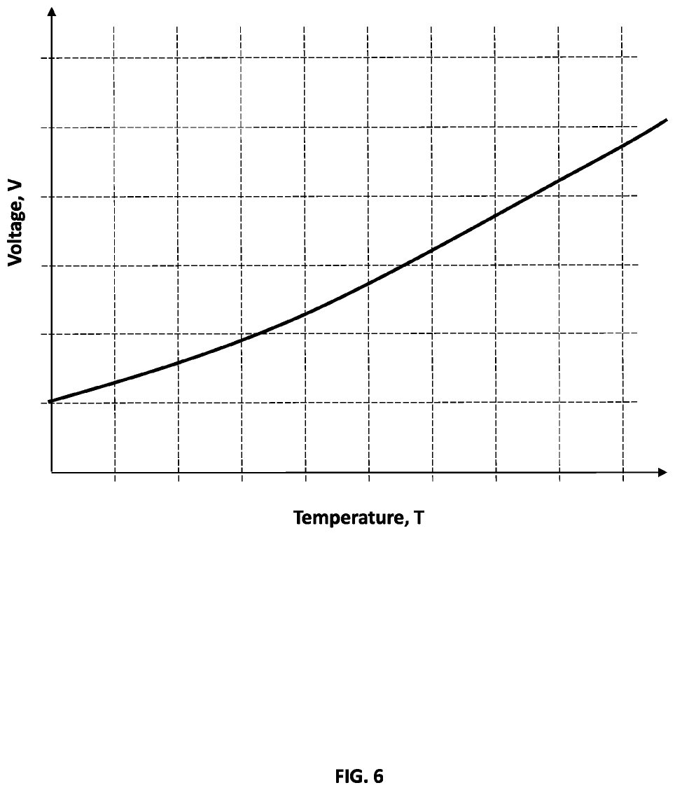

FIG. 6 provides a graphical representation showing an example of how the voltage, V associated with the temperature sensor may vary with temperature.

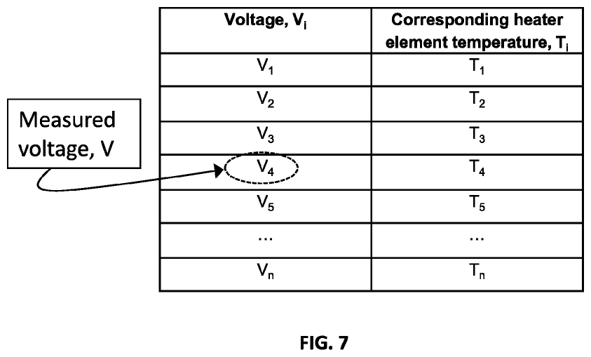

FIG. 7 illustrates an exemplary representation of a pre-configured look-up table of voltage and corresponding temperature values for the temperature sensor, and visually indicates how a measured voltage associated with the temperature sensor may be correlated with the pre-configured data to enable determination of heater element temperature.

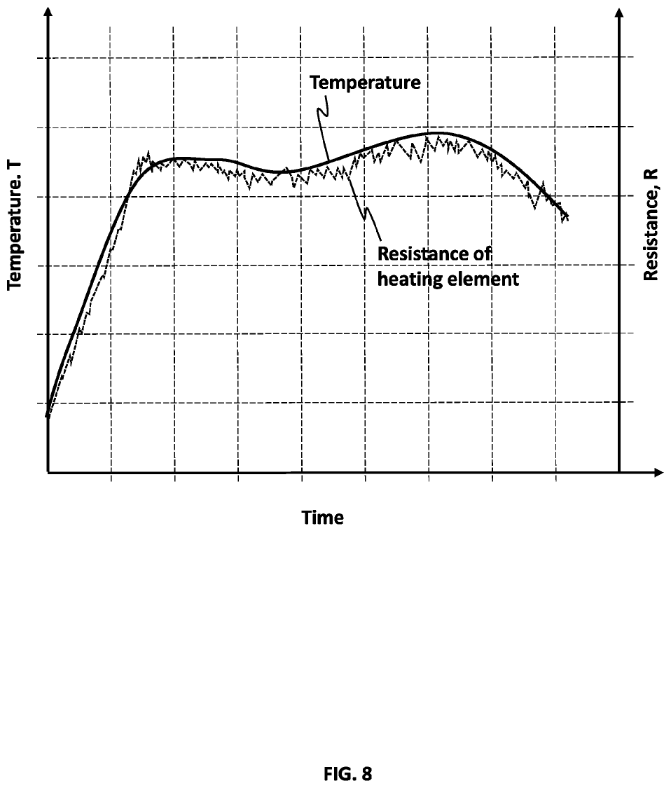

FIG. 8 illustrates an exemplary representation of the correlation between the impedance or resistance of the heating element of the device and the temperature of the heating element over a notional usage session.

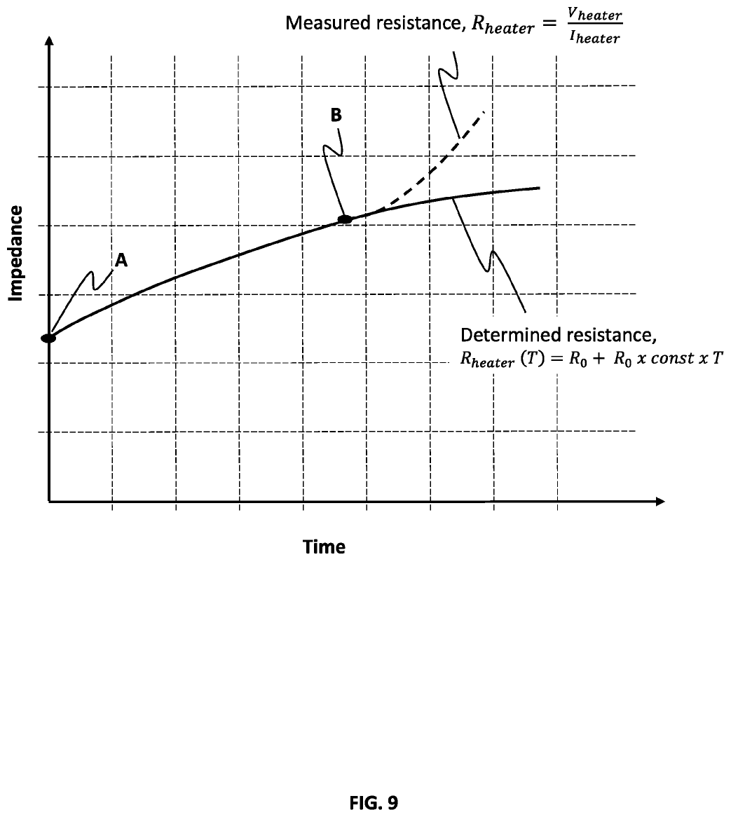

FIG. 9 shows an exemplary representation of the variation with time of both i) a measured impedance of the heating element and ii) an impedance of the heating element as determined from the temperature of the heating element as sensed by the temperature sensor.

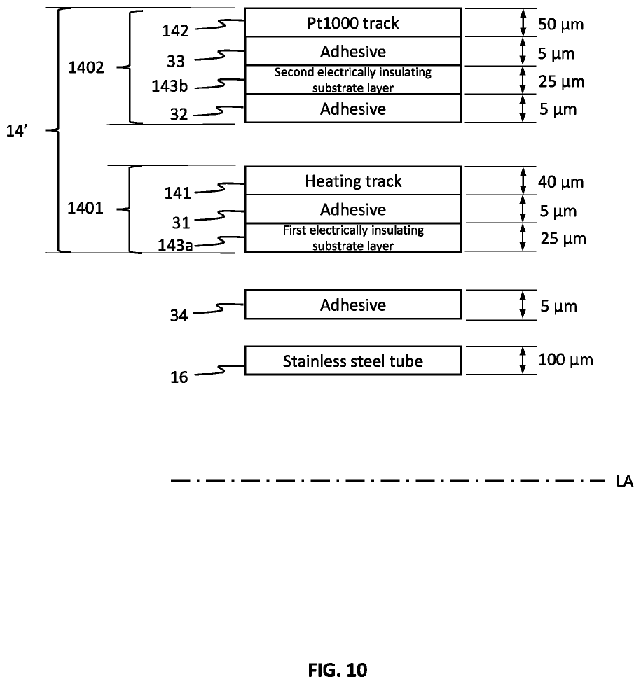

FIG. 10 shows a radial section view of a second embodiment of a heating assembly suitable for use in the aerosol-generating device of FIG. 1.

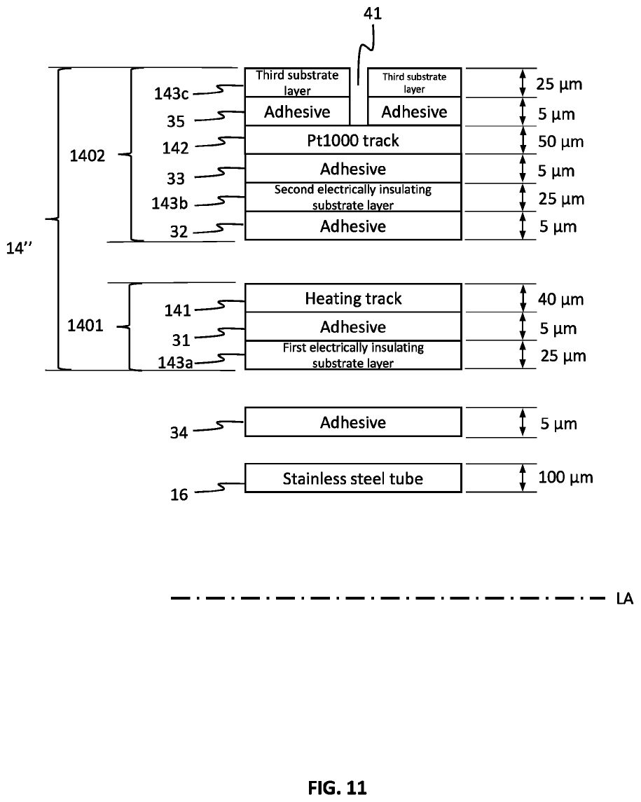

FIG. 11 shows a radial section view of a third embodiment of a heating assembly suitable for use in the aerosol-generating device of FIG. 1.

It is to be understood that the above figures are schematic representations and are not to scale.

FIG. 1 shows an aerosol-generating system 1 formed by a combination of an aerosol-generating device 10 and an aerosol-generating article 50 containing an aerosol-forming substrate 51. The aerosol-generating device 10 has an elongate housing 11 containing a power source 12, a controller 13 and a heating assembly 14. The controller 13 includes a microprocessor and related control electronics. The power source 12 is a rechargeable power source, in the form of a rechargeable battery. The heating assembly 14 is generally tubular in form and circumscribes a tubular wall of a cavity 15 of the aerosol-generating device 10. The tubular heating assembly 14 is formed of various components, including a resistive heating element 141 and a temperature sensor 142 (see FIGS. 2 and 3). The construction of the heating assembly 14 is described in more detail in subsequent paragraphs by reference to FIGS. 2 and 3.

FIG. 2 shows a view of a first embodiment of the heating assembly 14 prior to being rolled into a tubular form. For the state shown in FIG. 2, the heating assembly 14 has a generally planar form. The heating assembly 14 has a substrate layer 143. The substrate layer 143 is electrically insulative. For the example shown and described in these figures, the substrate layer 143 is made from polyimide; however, alternative electrically-insulative materials may be chosen for the substrate layer 143. The substrate layer 143 is flexible. The substrate layer 143 has first and second portions 144, 145 located adjacent to each other; the dashed line in FIG. 2 represents the interface between the first portion 144 and the second portion 145 of the substrate layer 143. The length, L of the substrate layer 143 is around two times the width, W of the substrate layer 143.

The heating element 141 is an electrically-resistive heating element and is provided as a sheet comprising a plurality of electrically conductive heating tracks (not shown). The heating element 141 is arranged on the first portion 144 of the substrate layer 143. First heating element contact area 146a and second heating element contact area 146b are arranged on the first portion 144 adjacent to and electrically coupled to the heating element 141. A first electrical contact 131a is provided contacting the first heating element contact area 146a. A second electrical contact 131b is provided contacting the second heating element contact area 146b. The first and second electrical contacts 131a, 131b are formed from electrical wire and are coupled to the controller 13. The controller 13 is coupled to the heating element 141 through contact between the first electrical contact 131a and the first heating element contact area 146a, and also through contact between the second electrical contact 131b and the second heating element contact area 146b. As shown in FIG. 1, the controller 13 is also coupled to the power source 12. The controller 13 is thereby able to control the supply of electric current to and through the electrically conductive tracks of the heating element 141.

A first temperature sensor contact area 147a and a second temperature sensor contact area 147b are arranged on the second portion 145 of the substrate layer 143. A third electrical contact 131c is provided contacting the first temperature sensor contact area 147a. A fourth electrical contact 131d is provided contacting the second temperature sensor contact area 147b. The third and fourth electrical contacts 131c, 131d are formed from electrical wire and are coupled to the controller 13. Third and fourth temperature sensor contact areas 147c, 147d are arranged on the surface of the second portion 145 of the substrate layer 143. The third temperature sensor contact area 147c is electrically connected to the first temperature sensor contact area 147a. The fourth temperature sensor contact area 147d is electrically connected to the second temperature sensor contact area 147b. The temperature sensor 142 is coupled between the third and fourth temperature sensor contact areas 147c, 147d. The temperature sensor 142 is a resistance temperature detector, such as a Pt100 or Pt1000 temperature sensor. However, in other embodiments, other forms of temperature sensor/resistance temperature detector may be employed.

The heating assembly 14 of FIG. 2 is rolled into a tubular shape and wrapped around a tube 16—as shown in FIG. 3. The longitudinal axis LA of the tube 16 extends into/out from the page for FIG. 3, with the width, W of the substrate layer 143 extending parallel to the longitudinal axis LA. The tube 16 defines the tubular wall of the cavity 15 of the aerosol-generating device 10. The tube 16 is formed of metal, such as stainless steel. However, in other embodiments, other metals or materials may be chosen for the tube 16. The heating assembly 14 is wrapped around the tube 16 such that the first portion 144 of the substrate layer 143 contacts the outer surface of the tube. The substrate layer 143 of the heating assembly 14 is wrapped over itself such that the second portion 145 of the substrate layer 143 defines an outward-facing surface of the tubular heating assembly 14, with the heating element 141 sandwiched between the first and second portions 144, 145.

Although not shown in FIG. 3, a glue layer or adhesive layer may be provided between the first portion 144 of the substrate layer 143 and the metal tube 16 to improve the connection between the substrate layer 143 and the tube 16. A further glue layer or adhesive layer may be provided between the first portion 144 of the substrate layer 143 and the second portion 145 of the substrate layer 143.

Once the heating assembly 14 is formed into the tubular shape shown in FIG. 3, the temperature sensor 142 is positioned on an outward-facing surface of the second portion 145 of the substrate layer 143. The temperature sensor 142 is arranged adjacent the heating element 141 at a position corresponding to a mid-point of the length L141 of the heating element, but distanced from the heating element 141 by the thickness of the second portion 145 of the substrate layer 143. In this manner, the temperature sensor 142 is thermally coupled to the heating element 141 and positioned so as to measure the hottest area of the heating element 141 during operation of the heating assembly 14 of the aerosol-generating device 10.

The aerosol-generating article 50 is inserted into the cavity 15 so that the tubular heating assembly 14 substantially encloses the entire length of the aerosol-forming substrate 51 of the article 50. The controller 13 is coupled to the power source 12 and the heating assembly 14 by electrical wiring. The controller 13 includes a memory module 131 containing a heating profile for a usage session of the aerosol-generating device 10. The heating profile defines a target operating temperature for the heating element 141 over the usage session. In use, the controller 13 controls the supply of electrical energy from the power source 12 to the heating element 141 in accordance with the heating profile stored in the memory module 131. The heating element 141 operates, under the control of controller 13, so as to heat the aerosol-forming substrate 51 of the aerosol-generating article 50 received in the cavity 15 and thereby generate an inhalable aerosol. A user draws directly on a mouth end 52 of the aerosol-generating article 50 to inhale the aerosol generated by the heating of the aerosol-forming substrate 51.

More specifically, the controller 13 controls the supply of electrical energy from the power source 12 to the heating element 141 so as to apply a voltage, Vₕₑₐₜₑᵣ across the heating element and a current, Theater through the heater element. This is shown schematically in FIG. 4. As the heating element 141 is an electrically resistive heating element and there are no or negligible inductive aspects present in the heating element 141, the impedance of the heating element may be regarded as being analogous to its resistance, Rₕₑₐₜₑᵣ. The controller 13 measures the impedance or resistance, Rₕₑₐₜₑᵣ of the heating element 141 based on knowledge of the voltage, Vₕₑₐₜₑᵣ and current, heater applied to the heating element. The controller 13 may itself measure the voltage, Vₕₑₐₜₑᵣ and the current, Theater applied to the heating element 141. The impedance or resistance, Rₕₑₐₜₑᵣ of the heating element 141 is related to the voltage, Vₕₑₐₜₑᵣ and the current, Iₕₑₐₜₑᵣ according to the following equation:

$${R}_{heater} = \frac{{V}_{heater}}{{I}_{heater}} \qquad Equation 3$$

As shown in FIG. 5, the temperature sensor 142 is coupled to a resistor 149. The temperature sensor 142 is coupled in series with the resistor 149. The resistor 149 has a known resistivity which is substantially invariant over the range of target operating temperatures for the heating element 141 defined in the heating profile stored in the memory module 131. In contrast, the resistivity of the temperature sensor 142 is variable with temperature. The relationship between resistance and temperature is known for the temperature sensor 142; such data may be provided by the manufacturer/supplier of the temperature sensor. The controller 13 controls the supply of electrical energy from the power source 12 so as to apply a voltage V_{T}, as shown in FIG. 5, thereby causing current flow, I through the resistor 149. The controller 13 is also configured to measure a voltage, V associated with the temperature sensor 142 (see FIG. 5). For the example illustrated in FIG. 5, the associated voltage, V being measured is the voltage across the temperature sensor 142. As indicated in the equation below, the relationship between the measured voltage, V and the resistance, R₁₄₂ of the temperature sensor 142 and the known resistance, R₁₄₉ of the resistor 149 is as follows:

$$V = \frac{{V}_{T} \cdot {R}_{142}}{{R}_{142} + {R}_{149}} \qquad Equation 4$$

So, by knowing voltages V and V_{T} and the temperature invariant resistance R₁₄₉ of the resistor 149, the resistance R₁₄₂ of the temperature sensor 142 may be determined. In turn, knowledge of the relationship between the resistance R₁₄₂ and temperature for the temperature sensor 142 means that it is possible to formulate pre-configured data correlating the measured voltage value, V, with the temperature of the temperature sensor 142.

FIG. 6 illustrates an exemplary correlation between the measured voltage, V and the temperature of the temperature sensor 142.

As shown in FIG. 7, the pre-configured data is provided in the form of a look-up table consisting of a plurality of voltage values, Vᵢ and corresponding temperature values, Tᵢ. The memory module 131 of the controller 13 stores this pre-configured data.

During operation of the aerosol-generating device 10, the controller 13 measures the voltage, V (as indicated in FIG. 5), and then correlates the measured voltage, V with the look-up table stored in the memory module 131. As illustrated in FIG. 7, the measured voltage, V is matched with the voltage value in the look-up table closest in magnitude to the value of the measured voltage—in this example, voltage value V₄. Based on the look-up table, the controller 13 determines the heating element 141 temperature to be the temperature value T₄ in the look-up table corresponding to voltage value V₄. In this way, the controller 13 is able to determine the heating element 141 temperature by correlating the voltage, V as measured by the controller 13 with the look-up table of voltage values, Vᵢ and corresponding temperature values, Tᵢ. The determined heating element 141 temperature may be referred to as the temperature as sensed by the temperature sensor 142.

The controller 13 then uses the determined heating element 141 temperature in combination with information provided by the heating element manufacturer to make a separate determination of the impedance or resistance, Rₕₑₐₜₑᵣ (T) of the heating element 141 according to the following equation:

$${R}_{heater} ( T ) = {R}_{0} + {R}_{0} \times const \times T \qquad Equation 5$$

The terms in Equation 5 above have the following meaning: T represents the determined heating element temperature; R₀ represents the resistance of the heating element 141 at a temperature T having a value of zero; “const” is a numerical constant whose value is dependent on the characteristics of the specific heating element employed in the aerosol-generating device 10 (for example, the material from which the heating element 141 is made) and is supplied by the manufacturer of the heating element. The above equation is illustrative of the correlation that exists between the impedance or resistance of the electrically resistive tracks of the heating element 141 and the temperature of the heating element. FIG. 8 illustrates an exemplary representation of the correlation between the impedance or resistance of the electrically resistive tracks of the heating element 141 and the temperature of the heating element over a notional usage session. As can be seen, the impedance or resistance of the heating element 141 closely tracks changes in the temperature of the heating element.

The controller 13 compares the measured impedance value, Rₕₑₐₜₑᵣ (corresponding to Equation 3) with the determined impedance value. Rₕₑₐₜₑᵣ (T) (corresponding to Equation 5). Where the magnitude of a difference between these two impedance values is greater than a predetermined threshold of 0.1 ohm, the controller 13 reduces the supply of power to the heating element 141. Exceeding of the predetermined threshold of 0.1 ohm is taken as an indicator that there may be a fault with the operation of the temperature sensor 142, or with the positioning of the temperature sensor 142 relative to the heating element 141. For example, the predetermined threshold may be exceeded where the temperature sensor 142 becomes detached from the heating assembly 14. Although a predetermined threshold of 0.1 ohm is used by the controller 13 for the embodiment described, in alternative embodiments higher or lower values may be used for the predetermined threshold. In a further alternative embodiment, the controller 13 is configured to reduce or terminate the supply of power to the heating element 141 dependent upon the measured impedance value Rₕₑₐₜₑᵣ (corresponding to Equation 3) differing in magnitude from the determined impedance value Rₕₑₐₜₑᵣ (T) (corresponding to Equation 5) by more than 1% of the determined impedance value. In a still further embodiment, the controller 13 is configured to reduce or terminate the supply of power to the heating element 141 dependent upon the determined impedance value Rₕₑₐₜₑᵣ (T) (corresponding to Equation 5) differing in magnitude from the measured impedance value Rₕₑₐₜₑᵣ (corresponding to Equation 3) by more than 1% of the measured impedance value. Higher or lower percentage levels may be employed by the controller 13.

FIG. 9 shows an exemplary schematic representation of the variation with time of the measured impedance, Rₕₑₐₜₑᵣ (corresponding to Equation 3) and the impedance as determined from the temperature of the heating element 141 as sensed by the temperature sensor, Rₕₑₐₜₑᵣ (T) (corresponding to Equation 5) when power is supplied to the heating element 141. Where the temperature sensor 142 is correctly positioned and correctly functioning, the plots for both measures of impedance generally overlap—this is represented by the region between points A and B in FIG. 9. Point B in FIG. 9 corresponds to an event in which the temperature sensor 142 becomes partially detached from the heating assembly 14. From this point forwards, if the controller 13 did not intervene, there would be a progressive divergence in the two impedance plots—this divergence is shown in FIG. 9. The divergence in the two impedance plots would occur due to the detachment of the temperature sensor 142 resulting in the sensor sensing temperatures which are lower than the actual temperature of the heater element 141. However, the configuration of the controller 13 of the aerosol-generating device 10 avoids and/or limits this divergence by reducing the supply of power to the heating element 141 when the divergence exceeds the predetermined threshold of 0.1 ohm. In a further embodiment, the controller 13 terminates the supply of power to the heating element 141 if the divergence exceeds a critical predetermined threshold, or if the act of reducing the supply of power to the heating element 141 does not reduce the divergence.

FIG. 10 shows a radial section view of a second embodiment of a heating assembly 14′. As for the heating assembly 14 of FIG. 3, the heating assembly 14′ of FIG. 10 is tubular once fully assembled. However, whereas the heating assembly 14 of FIGS. 2 and 3 uses a single substrate layer 143 of electrically insulative material, the heating assembly 14′ of FIG. 10 employs distinct first and second substrate layers 143a, 143b of electrically insulative material. The distinct electrically insulative substrate layers 143a, 143b are both made from polyimide; however, in other embodiments, other materials may be chosen for the substrate layers 143a, 143b. The heating assembly 14′ is fabricated by combining a first heating sub-assembly 1401 with a second heating sub-assembly 1402.

The first electrically insulative substrate layer 143a, a first adhesive layer 31, and the tracks of the heating element 141 are sequentially laid over each other to form the first heating sub-assembly 1401.

A second adhesive layer 32, the second electrically insulative substrate layer 143b, a third adhesive layer 33, and the temperature sensor 142 are sequentially laid over each other to form the second heating sub-assembly 1402.

The first and second heating sub-assemblies 1401, 1402 are brought together and adhere to each other by the adhesive action of the second adhesive layer 32, thereby forming the heating assembly 14′.

The heating assembly 14′ is wrapped around and adheres to the stainless steel tube 16 by the use of a fourth adhesive layer 34 there-between.

FIG. 10 illustrates the relative positions of each of the layers which form the heating assembly 14′ relative to the longitudinal axis LA.

FIG. 10 indicates exemplary thicknesses of each of the discrete layers which form the heating assembly 14′, as well as of the tube 16. The thickness of each of these elements is as follows: the tube 16 is of 100 micrometres thickness; the first and second electrically insulative substrate layers 143a, 143b are each of 25 micrometres thickness; the first, second, third and fourth adhesive layers 31, 32, 33, 34 are of 5 micrometres thickness; the heating element 141 is of 40 micrometres thickness; and the temperature sensor 142 is of 50 micrometres thickness.

FIG. 11 shows a view of a third embodiment of a heating assembly 14″. The heating assembly 14″ of FIG. 11 includes all of the features of the heating assembly 14′ of FIG. 10, but additionally includes a distinct third substrate layer 143c of electrically insulative material. In common with the distinct first and second substrate layers 143a, 143b, the third substrate layer 143c is formed from polyimide; however, in other embodiments, other materials may be chosen for the substrate layer 143c. The third substrate layer 143c (being of 25 micrometres thickness) is applied over the temperature sensor 142 by use of a fifth adhesive layer 35 (being of 5 micrometres thickness). However, a through hole 41 is provided through the thickness of the third substrate layer 143c to allow the temperature sensor 142 to be electrically coupled to the controller 13. The third substrate layer 143c is also thermally insulative, with the thermally insulative properties helping to reduce the likelihood of a user's fingers from being exposed to excessively high temperatures when holding the aerosol-generating device 10.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number “A” is understood as “A”±10% of “A”. Within this context, a number “A” may be considered to include numerical values that are within general standard error for the measurement of the property that the number “A” modifies. The number “A”, in some instances as used in the appended claims, may deviate by the percentages enumerated above provided that the amount by which “A” deviates does not materially affect the basic and novel characteristic(s) of the claimed invention. Also, all ranges include the maximum and minimum points disclosed and include any intermediate ranges therein, which may or may not be specifically enumerated herein.

1.-25. (canceled)

26. An aerosol-generating device for generating an aerosol from an aerosol-forming substrate, the aerosol-generating device comprising:
an electrically powered heater element configured to heat the aerosol-forming substrate;
a temperature sensor coupled to the electrically powered heater element and configured to sense a temperature of the electrically powered heater element;
a power source configured to supply power to the electrically powered heater element and the temperature sensor; and
control electronics configured to:
measure an impedance value of the electrically powered heater element,
correlate the measured impedance value with a value of impedance determined from the temperature of the electrically powered heater element as sensed by the temperature sensor, and
control the supply of power to the electrically powered heater element based on the correlation.

27. The aerosol-generating device according to claim 26, wherein the measured impedance value is derived from measurements of a voltage and a current applied to the electrically powered heater element.

28. The aerosol-generating device according to claim 26, wherein the control electronics are further configured to:
compare the measured impedance value with the determined impedance value, and
reduce or terminate the supply of power to the electrically powered heater element if a magnitude of a difference between the measured impedance value and the determined impedance value exceeds a predetermined threshold.

29. The aerosol-generating device according to claim 28, wherein the predetermined threshold is between 0.05 ohm and 1 ohm, or between 0.05 ohm and 0.5 ohm, or between 0.05 ohm and 0.2 ohm, or between 0.1 and 0.15 ohm.

30. The aerosol-generating device according to claim 26, wherein the control electronics are further configured to:
compare the measured impedance value with the determined impedance value, and
reduce or terminate the supply of power to the electrically powered heater element if the measured impedance value differs in magnitude from the determined impedance value by more than 5% of the determined impedance value, or by more than 2.5% of the determined impedance value, or by more than 1% of the determined impedance value, or by more than 0.5% of the determined impedance value.

31. The aerosol-generating device according to claim 26, wherein the control electronics are further configured to:
compare the measured impedance value with the determined impedance value, and
reduce or terminate the supply of power to the electrically powered heater element if the determined impedance value differs in magnitude from the measured impedance value by more than 5% of the measured impedance value, or by more than 2.5% of the measured impedance value, or by more than 1% of the measured impedance value, or by more than 0.5% of the measured impedance value.

32. The aerosol-generating device according to claim 26,
wherein the temperature sensor has a resistivity dependent on the temperature of the electrically powered heater element, and
wherein the control electronics are further configured to measure a voltage associated with the temperature sensor, the voltage being dependent on the resistivity of the electrically powered heater element, the control electronics comprising or being communicably coupled to a memory storing preconfigured data, the preconfigured data comprising a plurality of voltage values and a corresponding plurality of temperature values, and
wherein the control electronics further configured to correlate the measured voltage with the preconfigured data and determine the temperature of the electrically powered heater element based on the correlation between the measured voltage and the preconfigured data.

33. The aerosol-generating device according to claim 32, wherein the control electronics are further configured to compare the determined temperature of the electrically powered heater element with a target temperature for the electrically powered heater element, and to adjust the supply of power from the power source to the electrically powered heater element so as to reduce any difference between the determined temperature of the electrically powered heater element and the target temperature for the electrically powered heater element.

34. The aerosol-generating device according to claim 32,
wherein the temperature sensor is electrically coupled to a resistor, the resistor having a resistivity substantially invariant with temperature over a predetermined temperature range, and
wherein the temperature sensor and the resistor collectively form at least part of a resistor divider.

35. The aerosol-generating device according to claim 26, wherein the temperature sensor and the electrically powered heater element are disposed on opposed surfaces of an electrically insulative substrate layer.

36. The aerosol-generating device according to claim 35, wherein the electrically insulative substrate layer comprises two or more sublayers.

37. The aerosol-generating device according to claim 35, wherein the electrically insulative substrate layer comprises a first portion and a second portion, the electrically insulative substrate layer being rolled into a tubular shape such that the electrically powered heater element is disposed between the first and the second portions of the electrically insulative substrate layer.

38. The aerosol-generating device according to claim 35, wherein the temperature sensor is disposed on an outward-facing surface of the electrically insulative substrate layer.

39. The aerosol-generating device according to claim 35, wherein the electrically powered heater element is disposed between distinct first and second electrically insulative substrate layers.

40. The aerosol-generating device according to claim 39,
wherein the temperature sensor is disposed between the second electrically insulative substrate layer and a third electrically insulative substrate layer, and
wherein the first electrically insulative substrate layer, the electrically powered heater element, the second electrically insulative substrate layer, the temperature sensor, and the third electrically insulative substrate layer are successively laid over each other.

41. The aerosol-generating device according to claim 40, wherein an adhesive is provided between each of the successive layers.

42. The aerosol-generating device according to claim 40, wherein a through hole is provided through a thickness of the third electrically insulative substrate layer to provide access to the temperature sensor.

43. A method of controlling an electrically powered heater element of an aerosol-generating device, the aerosol-generating device comprising a temperature sensor coupled to the electrically powered heater element, the method comprising:
measuring an impedance value of the electrically powered heater element;
correlating the measured impedance value with a value of impedance determined from the temperature of the electrically powered heater element as sensed by the temperature sensor; and
controlling a supply of power to the electrically powered heater element based on the correlation.

44. The method according to claim 43, wherein the measuring the impedance value of the electrically powered heater element comprises measuring a voltage and a current applied to the electrically powered heater element.

45. The method according to claim 43, further comprising:
comparing the measured impedance value with the determined impedance value; and
reducing or terminating the supply of power to the electrically powered heater element if a magnitude of a difference between the measured impedance value and the determined impedance value exceeds a predetermined threshold.

46. The method according to claim 45, wherein the predetermined threshold is between 0.05 ohm and 1 ohm, or between 0.05 ohm and 0.5 ohm, or between 0.05 ohm and 0.2 ohm, or between 0.1 and 0.15 ohm.

47. The method according to claim 43, further comprising:
comparing the measured impedance value with the determined impedance value; and
reducing or terminating the supply of power to the electrically powered heater element if the measured impedance value differs in magnitude from the determined impedance value by more than 5% of the determined impedance value, or by more than 2.5% of the determined impedance value, or by more than 1% of the determined impedance value, or by more than 0.5% of the determined impedance value.

48. The method according to claim 43, further comprising:
comparing the measured impedance value with the determined impedance value; and
reducing or terminating the supply of power to the electrically powered heater element if the determined impedance value differs in magnitude from the measured impedance value by more than 5% of the measured impedance value, or by more than 2.5% of the measured impedance value, or by more than 1% of the measured impedance value, or by more than 0.5% of the measured impedance value.

49. The method according to claim 43,
wherein the temperature sensor has a resistivity dependent on the temperature of the electrically powered heater element,
the method further comprising:
measuring a voltage associated with the temperature sensor, the voltage being dependent on the resistivity of the electrically powered heater element,
correlating the measured voltage with preconfigured data comprising a plurality of voltage values and a corresponding plurality of temperature values, and
determining the electrically powered heater element temperature based on the correlation.

50. The method according to claim 49, further comprising:
comparing the determined temperature of the electrically powered heater element with a target temperature for the electrically powered heater element; and
adjusting a supply of power to the electrically powered heater element so as to reduce any difference between the determined temperature of the electrically powered heater element and the target temperature for the electrically powered heater element.
